(19) United States
(12) Patent Application Publication
(10) Pub. No.: US 2010/0137146 A1
(43) Pub. Date: Jun. 3, 2010

(54) Method of Screening Antibacterial Drug Compounds

(75) Inventors: Ramanujam Srinivasan, Singapore (SG); Mohan Balasubramanian, Singapore (SG); Maki Hori, Singapore (SG)

Correspondence Address: ROTHWELL, FIGG, ERNST & MANBECK, P.C., 1425 K STREET, N.W., SUITE 800, WASHINGTON, DC, 20005, US

(73) Assignee: Temasek Lifesciences Laboratory, Singapore (SG)

(21) Appl. No.: 12/305,523

(22) PCT Filed: Jun. 22, 2007
(86) PCT No.: PCT/SG2007/000175

§ 371 Date: Apr. 22, 2009

Related U.S. Application Data
(60) Provisional application No. 60/815,605, filed on Jun. 22, 2006.

(51) Int. Cl. C40B 30/00; C12Q 1/18; C12N 1/20

(52) U.S. Cl. 506/7; 435/32; 435/253.6

(57) ABSTRACT

Compounds are identified simultaneously as having antibiotic activity targeting a specific microbial protein and having no or limited toxicity against eukaryotic cells by expressing the microbial protein in eukaryotic cells by expressing the microbial protein in eukaryotic cells which then are used to screen candidate antibiotic compounds. Preferably, yeast cells such as Schizosaccharomyces pombe are transfected with and express a target bacterial protein such as FtsZ or MreB, optionally as a fusion with a reporter protein, and these transfected cells are used to screen libraries of compounds simultaneously for activity against the bacterial protein and lack of toxicity against the yeast cell.

This application claims the benefit of U.S. provisional application Ser. No. 60/815,605, filed Jun. 22, 2006, the entire contents of which is hereby incorporated by reference.

### BACKGROUND OF THE INVENTION

1. Technical Field

The invention relates to the fields of medicine and agriculture, and specifically to screening methods to identify antibiotic compounds that have activity to combat bacteria in vitro, in vivo in patients such as mammals, including humans, and in planta.

2. Description of the Background Art

Bacteria remain the greatest cause of infectious disease. Bacterial diseases result in both considerable human morbidity and mortality, and in tremendous losses in agriculture due to diseases of plants and animals. Decades after the discovery and extensive use of antibiotics, bacteria, particularly those that have acquired resistance to conventional antibiotics, have re-emerged as major threats. The discovery of new classes of anti-bacterial compounds is needed to control these new threats.

To date, almost all screens designed to identify antibacterial compounds have been performed using either bacterial cultures or purified bacterial proteins in vitro. While useful, these screens suffer from a major disadvantage in that they cannot identify drug candidates that are selectively toxic to bacteria and also have low or no substantial toxicity to human and/or plant tissues. Using in vitro bacterial culture or protein-testing methods to identify inhibitors or toxins of bacterial proteins therefore often also results in identification of compounds that are unsafe for use in eukaryotes such as human or animal patients or in agricultural crops. A second major disadvantage of the prior in vitro studies is that the identified compounds often do not function well enough in vivo to inhibit bacteria under physiological conditions. In any case, all the compounds identified in such assays must also be tested again for safety and effectiveness in eukaryotes. These two factors have limited the overall success rate of in vitro antibiotic screens.

The recently described bacterial cytoskeletal proteins FtsZ and MreB are conserved across all known bacterial species and are essential for the survival of the bacterium. FtsZ and MreB are essential genes that are highly conserved and ubiquitous in bacteria, including many pathogenic bacterial species. However, because FtsZ and MreB are structurally similar to eukaryotic tubulin and actin (which also are highly conserved across eukaryotes, e.g. yeasts, humans and plants), traditional screening methods directed at genes such as FtsZ and MreB also would identify a large proportion of compounds that would be toxic to animals and plants. It therefore would be necessary to expend considerable additional time and effort to rescreen each identified compound for toxicity in eukaryotes.

Therefore, there is a need in the art for convenient screening methods to identify antibacterial compounds which overcome the disadvantages of previous methods and also preferably identify antibacterial compounds targeted against new classes of bacterial genes.

### SUMMARY OF THE INVENTION

Accordingly, embodiments of this invention include a method of screening candidate antibacterial compounds for specific bacterial toxicity and simultaneously assessing the toxic effects the compounds may have to eukaryotic cells.

One embodiment of the invention provides an in vitro method for identifying antibacterial compounds that have significantly greater toxicity to a specific bacterial protein than to eukaryotic cells, which comprises providing a eukaryotic cell culture comprising eukaryotic cells, transfecting these cells with a gene encoding the bacterial protein, expressing the bacterial protein in the cells, screening these transfected eukaryotic cells with at least one candidate antibacterial compound for toxicity against or inhibition of the bacterial protein and simultaneously assessing the transfected eukaryotic cells for indications of toxicity against eukaryotic cells, thereby identifying a compound that exhibits a selective toxic effect against the specific bacterial protein. As used herein, toxicity to or toxic effects against the expressed bacterial protein means inhibition of or interference with a natural function or characteristic of the protein. In preferred embodiments, the eukaryotic cells are yeast cells, for example Schizosaccharomyces pombe or mammalian cells, for example rat kidney cells. Preferably, the bacterial protein is FtsZ, MreB or both FtsZ and MreB, which may be a fusion with a reporter protein such as Green Fluorescent Protein.

In another embodiment, the invention provides a method of identifying antibacterial target proteins from bacteria which comprises (a) providing a GFP fusion genomic library of a bacterium in a yeast expression vector; (b) expressing the library in yeast cells; (c) observing the yeast cells by fluorescence microscopy to identify GFP-labeled proteins of the expressed library that form visible assemblies; (d) assessing the identified proteins with respect to whether the proteins are essential to cell function of the bacterium; and (e) identifying proteins that form visible assemblies and are essential to cell function of the bacterium as antibacterial target proteins.

### BRIEF DESCRIPTION OF THE DRAWINGS

FIGS. 1A and 1B are photographs of wild-type Schizosaccharomyces pombe yeast cells stained to show F-actin (FIG. 1A) and tubulin (FIG. 1B).

FIGS. 2A and 2B are photographs showing Schizosaccharomyces pombe cells expressing a fusion of bacterial MreB with Green Fluorescent Protein.

FIGS. 3A and 3B are photographs of cells as in FIGS. 2A and 2B which have been treated with A22, an inhibitor of bacterial cell division in Gram-negative bacteria.

FIGS. 4A and 4B are photographs of cells as in FIGS. 3A and 3B, from which the A22 has been washed away.

FIG. 5 is a flow chart presenting a proposed model for MreB filament assembly and bundling.

FIGS. 6A and 6B are photographs of Schizosaccharomyces pombe cells carrying an FtsZ-GFP plasmid (6A) or both an FtsZ-GFP and a SulA plasmid (6B).

FIG. 7 is a photograph showing GFP-MreB localization in wild type S. pombe cells.

FIG. 8 is a photograph showing localization of mRFP-MreB in wild type S. pombe cells.

FIGS. 9A-9C are photographs showing localization of EcMreB (9A), tubulin (9B) or both EcMreB and tubulin (9C).

FIGS. 10A-10C are photographs showing localization of EcMreB (10A), actin (10B), or both EcMreB and actin (10C) after treatment with latrunculin A (LatA).

FIGS. 11A-11C are photographs showing localization of EcMreB (11A), actin (11B) or both EcMreB and actin (11C) after treatment with methyl 1-(butylcarbanoyl)-2-benzimidazolecarbamate (MBC).

FIGS. 12A-12L are a series of photographs taken at the indicated time intervals, with fluorescent labeling to show MreB in the simultaneous presence of LatA and MBC.

FIGS. 13A-13B are a pair of photographs of yeast cells treated with A22 which express GFP-MreB. FIG. 13A was taken 0 minutes after A22 wash; 13B was taken 20 minutes after A22 wash.

FIGS. 14A-14B are a pair of photographs of yeast cells displaying prominent GFP-MreB bundles, without (14A) and with 1 hour treatment with A22 (14B).

FIGS. 15A-15B are photographs of wild type yeast transduced with GFP-MreB E143A (15A) or GFP-MreB D165E (15B) to introduce a mutation in MreB.

FIG. 16 (1-24) is a series of photographs showing yeast cells expressing GFP-MreB, treated with A22, after removal of A22. The photograph's were taken at 2-minute intervals. Vertical lines show the position of the cell nucleus.

FIGS. 17A-17D are photographs taken at the indicated times after photobleaching. The white box indicates the bleached region; the darker box indicates the unbleached area.

FIG. 18 is a bar-graph showing growth percentage of MreB bundles on either sides of the bleached region of FIG. 17.

FIG. 19 shows recovery of fluorescence intensity after bleaching as shown in FIG. 17.

FIGS. 20A-20B show fluorescence recovery data for the bleached (20A) and unbleached (20B) regions.

FIGS. 21A-21B are time-lapse studies of MreB polymer growth after treatment with A22 and its subsequent removal.

FIGS. 22A-22F are two sets of three photographs showing localization of EcMreB (22A, 22D), tubulin (22B, 22E) and both Ec MreB and tubulin (22C, 22F) in teal cells.

FIGS. 23A-23B are photographs of GFP-MreB transduced weel-50 cells, unstained (23A) and stained for GFP-MreB (22B).

FIGS. 24A-24E are photographs of mutant yeast cells (24A: arp2; 24B: arp3; 24C: cdc12; 24D: for3; 24E: alp4) expressing GFP-MreB and stained for MreB and actin.

FIGS. 25A-25G are a time-lapse study of MreB bundle growth in wee1-50 cells.

FIGS. 26A-26F are photographs showing the organization of scrambled arrays of microtubules and GFP-MreB in orb6-25 cells.

FIG. 27 is a photograph of normal rat kidney (NRK) cells, expressing GFP-MreB.

FIG. 28 is a map of pEGFP-C1.

### DESCRIPTION OF EMBODIMENTS OF THE INVENTION

The screening methods disclosed herein involve expressing genes that encode bacterial proteins in a eukaryotic organism, which then is used to test or screen candidate antibacterial compounds that target the expressed protein. The eukaryote carrying the target bacterial protein can be screened by contacting a culture of the eukaryote, in vitro, with compounds and identifying those that simultaneously exhibit the desired toxic action on the expressed bacterial protein and are non-toxic to the eukaryote. Any compound may be screened by this method. Thus, such screens in a eukaryotic cell culture, such as a yeast culture, have the added benefit that it is possible simultaneously to detect and eliminate compounds that may be toxic to bacteria, but also negatively affect or kill the eukaryotic cell. Molecules identified in the screens of embodiments of the present invention therefore are specifically targeted against bacteria and unlikely to negatively affect eukaryotic cells such as mammalian cells, plant cells and the like in use in vivo. Thus, screening methods according to embodiments of the invention can be used to ensure that the compounds identified in the screen are already pre-screened and evaluated for toxicity to eukaryotic cells in the first instance, saving valuable time and resources that are lost when numerous useless toxic compounds have to be re-tested for safety in animals or plants as required in prior art methods.

These methods also allow screening for and discovery of compounds which are targeted against new bacterial proteins. Such antibacterial compounds would be members of an entirely new class of antibiotics. Using the method, large banks or libraries of chemicals can be screened against new bacterial proteins to discover entirely new classes of antibacterial drugs. Any known screening method that tests for toxic effects on the expressed bacterial protein and can be performed on eukaryotic cells in culture may be suitable as a screening method for use with this invention. Conveniently, the compounds may be added to the medium culturing the eukaryotic cells and the cells observed, both for effects on the cell itself and on the expressed target bacterial protein. A small molecule or peptide library can be screened in this way to identify compounds that are toxic to or negatively effect (e.g. inhibit the function of) the expressed bacterial protein(s).

Testing for toxic effects on the eukaryotic cell advantageously can be performed as part of the same screen or concomitantly with the bacterial toxicity screen but in a second step performed before or after bacterial toxic effcts are assessed. Screening for effects on the eukaryotic cells can be by simple observation of the cells during the screen to detect negative effects, for example cytotoxicity, that are visually apparent. As well, the screens for eukaryotic effects can include monitoring any visible morphological changes in cell shape, monitoring growth rates, performing viability assays and the like, including any biochemical or morphological assay.

Eukaryotic cells that are suitable for expression of a bacterial target protein and screening include yeast, mammalian, plant, insect or any eukaryotic cell type for which a convenient cell culture and transfection technology exists. Yeast cells generally are preferred because of their ready availability, well-understood nutritional requirements and metabolic pathways and ease of culture. Schizosaccharomyces pombe are suitable yeast cells, however insect cell lines such as Sf21, mammalian cell lines such as HeLa and plant cell lines such as BY2 (tobacco) and the like also are suitable.

An important initial step toward developing a new anti-bacterial compound is the identification of a target for the compound. FtsZ and MreB are preferred bacterial target proteins for use in the invention, in part because they constitute drug targets that, at present, are not the targets of any commercially available antibiotics, and therefore can be used to discover entirely new classes of antibiotic compounds. Such compounds would be highly valuable against multi-drug resistant strains of pathogens, which are becoming prevalent. Further, the fact that these proteins are highly conserved among almost all bacterial species, including Gram-positive and Gram-negative pathogens of both humans and plants, allows one to identify compounds that likely will have a very broad spectrum of effectiveness across many types of bacteria. Thus, compounds which inhibit these proteins have the potential to represent an important new class of broad spectrum antibiotics. The methods are equally applicable, however, to any potential target of an antibacterial compound.

Additional examples of bacterial proteins that are suitable for use with the invention include any bacterial protein target that is known presently or subsequently discovered. Cytoskeletal proteins and any other bacterial proteins that form polymers and are essential for the survival of the bacterium constitute examples of preferred new drug targets which may be used with embodiments of the invention. To discover new antibiotic target proteins, a bacterial genomic library may be constructed as a GFP or other reporter, fusion in a yeast expression vector, for example. Fluorescence microscopy then can be used to identify proteins which form visible polymeric assemblies or structures in the eukaryote. Any identified proteins then can be assessed further with respect to whether they are essential to bacterial cell function. A protein is considered essential, or essential to bacterial cell function, if bacterial cells die, fail to grow (multiply) or growth (multiplication) is severely curtailed when the protein or an antibacterial functional equivalent thereof is greatly reduced or substantially absent. If the protein is essential, it is a useful protein target for identifying antibacterial drugs according to the methods of this invention. Such protein targets thus are examples of proteins that can be used according to embodiments of the invention to screen libraries of compounds for toxicity to the target protein and thus bacteria.

The invention contemplates screens that test for specific bacterial toxicity as described herein against any bacterial protein target, however preferred bacterial protein targets for screening are those which are conserved across bacterial species, and preferably are either essential to one or more required bacterial function or are necessary for bacterial survival or cell division. Additional bacterial protein targets that can be used with-embodiments of the invention therefore include, but are not limited to other proteins that form molecular assemblies that can be visualized by microscopy, e.g. Crescentin of Caulobacter crescentus or its orthologs from Helicobacter pylori. Other examples of suitable protein targets include SopA and ParM from E. coil. One or more than one protein may be expressed in the eukaryotic cells in different embodiments of the invention.

The bacterial proteins used according to the invention may be expressed in the eukaryote in any form suitable for an assay of toxicity to their function. In some embodiments, the protein is expressed as a fusion of the full-length bacterial protein with a reporter to facilitate identification, quantitation and/or localization of the bacterial protein. Those of skill in the art are aware of many reporter genes that can be fused to the gene expressing the protein of interest, including, for example, LacZ or other small tags such as HA, myc and the like that would permit visualization of the protein in terms of cellular localization. Any of these known reporter genes or proteins are contemplated for use with the inventive methods here. Preferably, when it is desirable to visualize the protein for detection or screening methods, the protein is expressed as a fusion with green fluorescent protein (GFP) for example, or in any manner allowing subsequent visualization. Of course, the bacterial protein may be visualized using specific antibody labeling according to well-known techniques in the art, as well.

Functional MreB and FtsZ both form polymeric filamentous structures in vitro and in vivo. These filaments are essential for bacterial chromosome segregation and maintenance of cell shape. Loss of function of either of these proteins prevents bacterial division and causes cell death. The polymeric forms of these proteins can be identified easily using fluorescence microscopy when the proteins are expressed as a fusion with GFP or the like, or expressed, for example as a polypeptide having a binding site which can be exploited to attach a detector molecule. Methods for achieving labeling of expressed proteins are well known in the art. Any of these is contemplated for use with the invention.

MreB organizes into linear bundles that grow in a symmetrically bidirectional manner at 0.46±0.33 μm/min, with new monomers and/or oligomers being added along the entire length of the bundle. Organization of linear arrays was dependent on the ATPase activity of MreB, and their alignment along the cellular long axis was achieved by sliding along the cortex of the cylindrical part of the cell. The cell ends appeared to provide a physical barrier for bundle elongation. See examples.

S. pombe is a eukaryotic cellular system useful to further study the functional properties of proteins such as the E. coli actin-related protein MreB. S. pombe was chosen as a model for the study of the bacterial proteins MreB and FtsZ in part because of its well-characterized actin and microtubule cytoskeletons, however any convenient eukaryotic cell may be used.

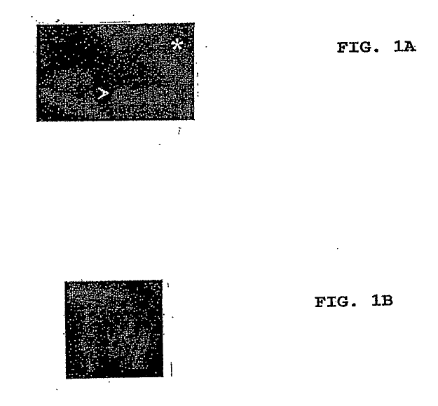

Wild-type S. pombe cells were fixed and stained with rhodamine-conjugated phalloidin to visualize F-actin, which is detected in patches concentrated at the cell ends and in linear cables during interphase, and as a medial ring in mitotic cells. FIG. 1A shows the organization of actin in wild-type S. pombe yeast. In the Figure, arrows point to F-actin patches present at a cell end, and an asterisk points to an F-actin ring. In wild-type interphase S. pombe cells fixed and stained with antibodies against tubulin, linear arrays of microtubules were observed in antiparallel arrays along the long axis. Tubulin was detected in an intranuclear spindle during mitosis. See FIG. 1B.

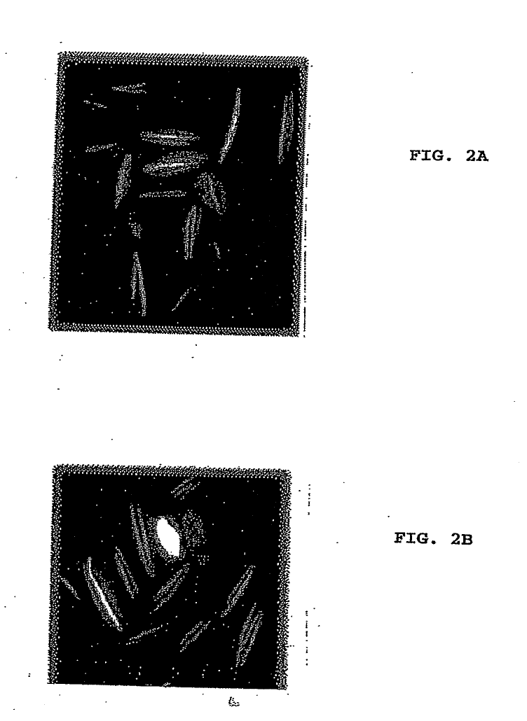

When expressed in yeast, both GFP-MreB and FtsZ-GFP fusion proteins formed characteristic bacterial polymeric structures. See FIGS. 2A and 2B, which are representative of the polymeric structures formed by MreB. Eukaryotic cells expressing a fusion of MreB or FtsZ and a fluorescent protein, such as these, therefore can be contacted with any compound to determine whether that compound possesses antibacterial effects without resulting in harm to the eukaryotic cell. A compound, or a library of compounds, can be screened and evaluated in S. pombe cells expressing GFP-MreB, for example, both for inhibition of polymerization and for toxicity to the eukaryotic cell.

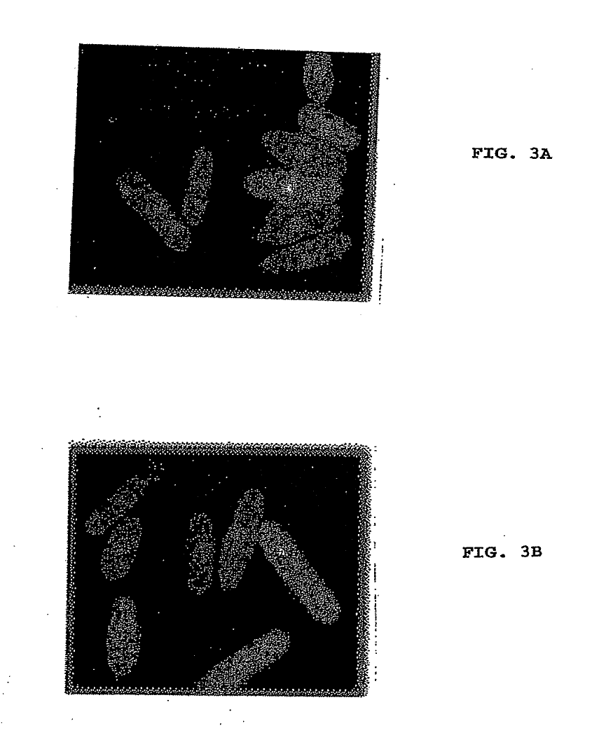

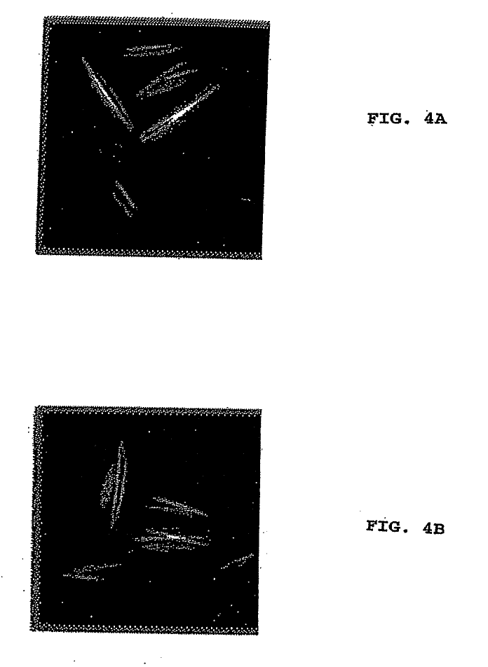

Inhibition of polymerization is easily assessed visually using a fluorescence microscope by observing a lack of filaments in the cells. Lack of toxicity to the cells also can be assessed at the same time. See FIGS. 3A and 3B, which are photographs of cells that express GFP-MreB as in FIG. 2, but which have been treated with A22, a compound that inhibits bacterial cell division in Gram-negative bacteria, to demonstrate loss of MreB function. Molecules identified as inhibiting the bacterial protein or its function, but not negatively affecting the eukaryotic cell, are specifically targeted against bacteria for which the tested protein is necessary for optimal survival. They would not be expected to negatively affect eukaryotes such as humans or plants in which the compounds would be used to combat bacteria. FIGS. 4A and 4B show cells from which the A22 has been washed away.

MreB did not co-distribute with tubulin (microtubules) and the MreB bundles remained unperturbed by treatment-with latrunculin and/or the microtubule inhibitor methyl-1-butylcarbamoyl-2-benzimidazolecarbonate even when microtubules were completely disassembled. This established that MreB assembled into linear filaments or bundles in fission yeast and that MreB assembly and maintenance was independent of F-actin and the microtubule cytoskeletons of the yeast. These new findings relating to the mechanism of MreB polymerization and organization are presented in FIG. 5, a proposed model for MreB filament assembly and bundling. Applicants do not wish to be bound by theory, however, under this model, ATP binding results in the formation of short filaments, which later bundle to form rigid linear polymers, and is likely to be concomitant with ATP hydrolysis. ATP hydrolysis is likely to trigger a conformational change in MreB protofilaments, which in turn might result in bundling. Mutations that block ATP hydrolysis thus result in proteins that do not assemble into rigid linear polymeric arrays.

The E. coli tubulin-related protein FtsZ assembles polymers in fission yeast. Its assembly is inhibited by co-expression of SulA, a known inhibitor of FtsZ polymerization. See FIG. 6. This allows the fission-yeast expression system to be generally useful for the study of the bacterial cytoskeleton. Expression in fission yeast also allows the cell-biological analyses of mutant versions of bacterial-cytoskeleton proteins, which cannot be studied in bacterial cells because of lethal consequences. Previous studies have investigated the cellular organization of MreB ATPase mutants in merodiploids, which also contain a wild-type copy of MreB. In contrast, the present system allows one to study and characterize MreB ATPase mutants in isolation. A more dramatic defect in the ability of these mutants to form filaments or bundles is evident.

Expression of E. coli FtsZ (EcFtsZ) as an FtsZ-GFP fusion in S. pombe is shown in FIGS. 6A (S. pombe carrying FtsZ-GFP-plasmid) and 6B (S. pombe carrying both pREP42-ftsZ-gfp (ura*) and pREP41-sulA-HA (leu*)). Cultures were grown at 30° C. in the absence of-thiamine to allow the expression of the proteins from the nmt promoter. Under these conditions, EcFtsZ formed cables and patches (or discs), and its formation was inhibited by the coexpression of its known inhibitor SulA. Compare FIGS. 6A and 6B.

In summary, therefore, preferred methods according to the invention involve transfer of a gene encoding a target bacterial protein, for example MreB, FtsZ or both, to a convenient eukaryotic cell in culture, for example a yeast cell such as S. pombe or a mammalian cell, which is then contacted in vitro with one or more candidate antibiotic compounds so that both bacterial and eukaryotic toxicity can be assessed, in one system, in the first instance. The target bacterial protein may be any bacterial protein which, when inhibited, can cause a bacterio-toxic result. The protein may be expressed as a fusion with another protein that can act as a reporter or a binding site for a label for protein identification. The bacterial proteins MreB and FtsZ are most preferred. Although any convenient eukaryotic cells or cell lines may be used, including for example insect, mammalian, human or any cells, yeast are preferred.

The following examples are included to illustrate preferred examples of the inventive methods and are not intended to be limiting.

### Examples

### Example 1

### Fusion-Protein Construction and Transformation of the Yeast Schizosaccharomyces pombe

Transformations with the plasmids were carried out by using the Lithium-acetate method on S. pombe strain MBY192 (h⁻ leu1-32 ura4-D18) (lab collection) or S. pombe mutants MBY241 (h⁻ arp3-1c leu1-32 ura4D-18 ade6-210), MBY311 (h⁻ cdc12-112 leu1-32 ura4D-18), MBY598 (h⁹⁰ arp2-1a mam2::leu2 leu1-32 ura4D-18 ade6-216), MB-Y1446 (h⁻ alp4-1891 leu1-32), MYB1565 (wee1-50 ura4D-18), MBY2527 (h⁻ Δfor3::kan leu1-32 ura4D18 ade6), MBY415 (h⁻ orb6-25 ade6-M210 leu1-32), and MBY3197 (h⁻ tea1Δ946::kanR leu1-32 ura4-D18 ade6-M216). GFP-MreB, its mutants, and FtsZ-GFP were all expressed from the medium-strength nmt42 promoter. The following fusions were constructed: gfp-mreB, mrfp-mreB, ftsZ-gfp, and sulA-HA.

The ORF corresponding to GFP was amplified from pREP42-GFP with primers MOH2411 and MOH2412 (for fusion to mreB) and MOH1017 and MOH1276 (for fusion to ftsZ). See Table I, below for primer sequences. The primers MOH2413 and MOH2414 (for mreB) and MOH2409 and MOH2410 (for ftsZ) were used to amplify the respective genes from E. coli genomic DNA. The MreB product was digested with Xbal and BamHl and ligated along with GFP product digested with Sall and Xbal in a three-way ligation to pREP42 (ura⁺) or pREP41 (leu⁺) digested with Sall and BamHl, resulting in a plasmid containing a N-terminal GFP fusion to MreB under the nmt42 promoter. The ORF corresponding to the monomeric verison of RFP was PCR amplified from pRSET-mRFP, with primers MOH2590 and MOH2591, and digested with Sall and Xbal. This product, along with MreB digested with Xbal and BamHl, was ligated to pREP42 digested with Sall and BamHl to obtain mRFP-MreB. For obtaining MreB under the CMV promoter, MreB was PCR amplified with primers MOH2581 and MOH2414, digested with EcoRl and BamHl, and ligated to pEGFP-C1 digested with the same enzymes, resulting in a N-terminal GFP fusion to MreB. The C-terminal fusion of GFP to FtsZ was generated by a three-way ligation of GFP product digested with Pstl and BamHl and FtsZ product digested with Ndel and Pstl to pREP42 digested with Ndel and BamHl. Primers MOH2506 and MOH2507 were used to amplify the SulA ORF. The PCR product was digested with Ndel and BamHl and ligated to pREP41-HA.

The MreB ATPase mutants were generated by overlap PCR with the following mutagenic primers: MOH2516 and MOH2517 for E143A and MOH2512 and MOH2513 for D165E along with the above-mentioned end primers (MOH2413 and MOH2414). The GFP-MreB mutants were all cloned into pREP42. All plasmids and mutations were verified by sequencing. The list of primers and sequences are given in Table I below.

TABLE I

Primers.

| Primer | | SEQ ID |
|---|---|---|
| Name | Sequence (5′ -> 3′) | NO. |
| MOH1017 | GTCGTCGGATCCTTATTTGTATAGTTCATCCATGC | 1 |
| MOH1276 | GCCTCGCTGCAGATGAGTAAAGGAGAAGAACTTTTC | 2 |
| MOH2409 | GGCGGCCATATGTTTGAACCAATGGAACTTAGCAATGAC | 3 |
| MOH2410 | TGGTCTGCAGGTTGTTGTTGTTATCAGCTTGCTTACGCAGGAATGCTGGGAT | 4 |
| MOH2411 | GTCGTCGTCGACCATGAGTAAAGGAGAAGAAC | 5 |
| MOH2412 | GTCGTCTCTAGATTTGTATAGTTCATCCATGC | 6 |
| MOH2413 | GCGGGCGTCTAGAATGTTGAAAAAATTTCGTCGcATG | 7 |
| MOH2414 | CCCCCCGGATCCTTACTCTTCGCTGAACAGGTCG | 8 |
| MOH2506 | GGCGGCCATATGTACACTTCAGGCTATGCACATC | 9 |
| MOH2507 | CCCCCCGGATCCTTAATGATACAAATTAGAGTGAATTTTTAG | 10 |
| MOH2512 | CGACCGGTTCTATGGTGGTTGAAATGGGTGGTGGTACCACTGAAG | 11 |
| MOH2513 | CTTCAGTGGTACCACCACCGATTTCAACCACCATAGAACCGGTCG | 12 |
| MOH2516 | GTGAAGTCTTCCTGATTGAAGCACCGATGGCTGCCGCAATTGG | 13 |
| MOH2517 | CCAATTGCGGGAGCCATCGGTGCTTCAATCAGGAAGACTTCAC | 14 |
| MOH2581 | CCGCCGAATTCTATGTTGAAAAAATTTCGTGGCATG | 15 |
| MOH2590 | GTCGTCGTCGACCATGGCCTCCTCCGAGGACGTCATC | 16 |
| MOH2591 | GTCGTCTCTAGAGGCGCCGGTGGAGTGGCGG | 17 |

The restriction sites or the nucleotide changes made for the mreB mutants incorporated in the primers are underlined.

### Example 2

### Expression of GFP Fusion Proteins and Fluorescence Microscopy

GFP-MreB and FtsZ-GFP were expressed in S. pombe by growing the transformed cultures in the absence of thiamine for 16-20 hours. In the case of fission-yeast temperature-sensitive mutants carrying the GFP-MreB plasmid, cultures were grown in the absence of thiamine for 25-30 hours at 24° C. to allow sufficient expression of the protein. However, prior to the formation of MreB-polymers, the cultures were shifted to restrictive temperatures for a period of 4 hours. The cells were subsequently fixed and stained for actin or tubulin.

The cells were-either fixed with formaldehyde 0.7%) or observed live under a fluorescence microscope. Inhibition of MreB polymerization was achieved by the addition of A22 at a final concentration of 50 μg/ml after 14-16 hours of growth in the absence of thiamine. Cultures were allowed to grow further for a period of 6-8 hours before washing of A22 for time-lapse microscopy. The cells were mounted onto agarose slides, and images were acquired at fixed time intervals. Immunofluorescence was carried out as described in Balasubramanian et al., Meth. Enzymol. 283:494-495, 1997.

S. pombe cells expressing GFP-MreB were fixed with methanol and incubated with tubulin (1:100) antibodies. Mouse secondary antibodies conjugated to Alexa-594™ were used at 1:400 for visualizing tubulin. For actin staining, cultures were fixed with formaldehyde (3.7%) and permeablized by using 1% Triton-X 100 in PBS. Rhodamine-conjugated phalloidin was used to visualize S. pombe actin. Latrunculin A (LatA) was added at a final concentration of 20 μg/ml for 10 minutes, and methyl 1-(butylcarbanoyl)-2-benzimidazolecarbamate (MBC) was added at a 25 μg/ml for 20 minutes. For methods, see Karagiannis et al., Mol. Biol. Cell 16:358-371, 2005 and Sawin et al., J. Cell Sci. 117:689-700, 2004, the disclosures of which are hereby incorporated by reference. For time-lapse imaging, LatA and MBC were included simultaneously in the agarose pads. For epiflourescence microscopy, the cells were observed with an Olympus™ IX71 microscope with a PLAN APO 100×/1.45 objective. Pictures were obtained with a CCD camera (CoolSnapHQ™, Photometrics™). The images were acquired and processed with Meta-morph™.

### Example 3

### Bacterial Actin Expression

The plasmid containing the GFP-MreB gene fusion (pREP42-gfp-mreB) was transformed into S. pombe cells using lithium acetate. The transformants were grown in the absence of thiamine for 18-22 hours to induce the expression of GFP-MreB. The polymeric structures formed by MreB were observed under a fluorescent microscope.

FIGS. 2A and 28 show MreB (a bacterial actin) expressed in S. pombe as a fusion with GFP. Filaments produced in the yeast are clearly visible.

### Example 4

### Visualization of the Distribution of MreB

To visualize the distribution of MreB, a GFP-MreB fusion protein was expressed in fission-yeast cells. Wild-type S. pombe cells were transformed with a plasmid that allowed expression of a GFP-MreB fusion. Cells were grown in medium lacking thiamine to allow maximal expression from the nmt42 promoter. Unlike F-actin in fission yeast, GFP-MreB was not detected in cell-end-localized patches or in medial rings. MreB assembled into linear arrays parallel to the long axis of the cell (FIG. 7). The spirals of MreB seen in E. coli were not observed in the yeast culture. The organization was more similar to that of the bacterial-plasmid-segregating proteins, ParM, and some versions of SopA.

Two major distribution patterns were observed. In 42.7% (n=1800) of the cells, a prominent thick bundles was found to run from one end of the cell to the other. See FIG. 7; cell marked with *. In 57.3% of the cells, two to four bundles were found to run from one end of the cell to the other. See FIG. 7; cells marked with arrowhead.

Similar linear bundles of MreB were also observed when a monomeric version of RFP (as described in Campbell et al., Proc. Natl. Acad. Sci. USA 99:7877-7882, 2002) was fused to MreB. See FIG. 8. For this study, wild-type S. pombe cells were transformed with a plasmid that allowed expression of an mRFP-MreB fusion. Cells were grown in medium lacking thiamine to allow maximal expression from the nmt42 promoter. Interestingly, MreB bundles did not curve around the hemispherical ends of the cell and appeared to stop growth upon reaching the cell tips and in many instances resembled the interphase microtubules of fission yeast. These data show that MreB Polymerization is independent of actin and microtubules.

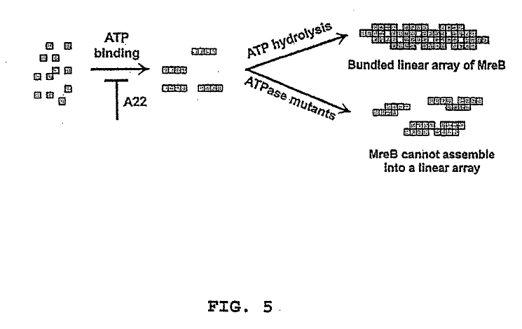

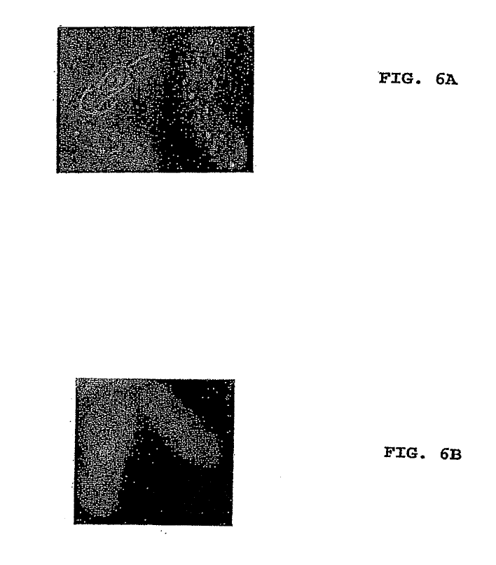

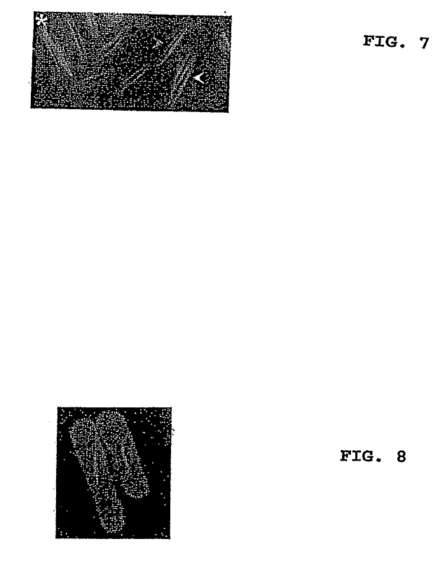

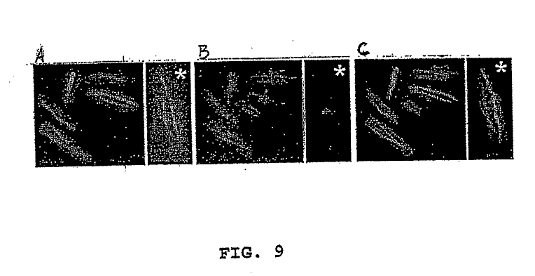

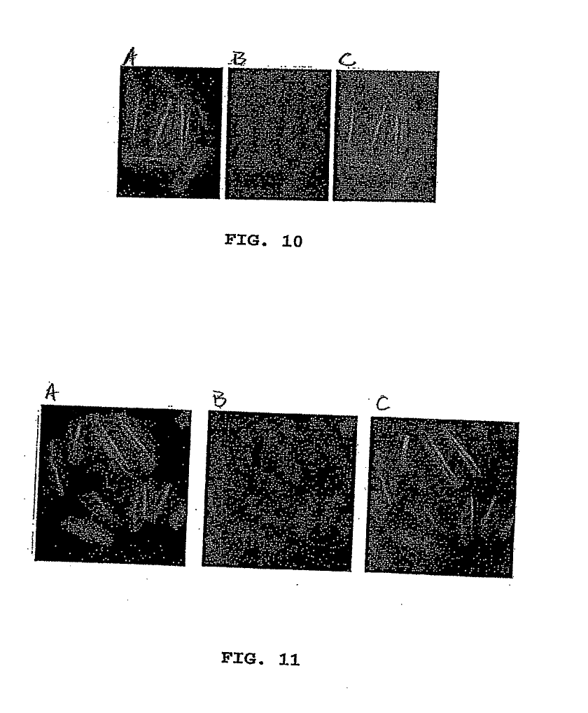

Colocalization of MreB with interphase microtubules was tested. Staining of GFP-MreB-expressing S. pombe cells with antibodies against tubulin established that GFP-MreB did not codistribute with microtubules. See FIG. 9 (A=EcMreB; B=Tubulin; C=Both). This conclusion was particularly clear in mitotic cells in which the MreB array was detected in the cytoplasm whereas the microtubules were organized into an intranuclear spindle. See FIG. 9 (an asterisk indicates a mitotic cell with an intranuclear spindle and cytoplasmic arrays of MreB).

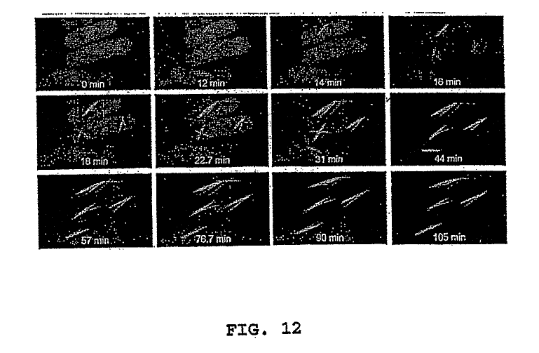

Although MreB did not codistribute with the microtubules or F-actin, it was possible that the maintenance of linear arrays of MreB was somehow dependent on their presence. Studies therefore were performed to determine whether MreB assembly and maintenance was dependent on F-actin and microtubules. Cells were grown in the absence of thiamine to allow expression GFP-MreB. Cultures were then treated with LatA (20 μg/ml) for 10 minutes or methyl 1-butylcarbamoyl-2-benzimidazolecarbamate MBC (25 μg/ml) for 20 minutes to disrupt F-actin and microtubule, respectively. Rhodamine-conjugated phalloidin was used to stain actin; microtubules were stained with tubulin antibodies. MreB bundles remained unperturbed even after treatment with latrunculin A (LatA) or the microtubule inhibitor (MBC), when actin and microtubules were completely disassembled. See FIGS. 10 (treatment with LatA; A=EcMreB; B=Actin; C=Both) and 11 (treatment with MBC; A=EcMreB; B=Actin; C=Both). To test the effect of a lack of both F-actin and microtubules, cells expressing GFP-MreB were grown in the presence of A22 (50 μg/ml). A22 was subsequently washed out, and after removal from A22, cells were mounted onto agarose pads containing both LatA (20 μg/ml) and MBC (25 μg/ml). Images were then acquired at 1-minute intervals. MreB nucleated and assembled into polymers even in the simultaneous presence of LatA and MBC. See FIG. 12.

### Example 5

### MreB Polymerization in Fission Yeast and Bacteria

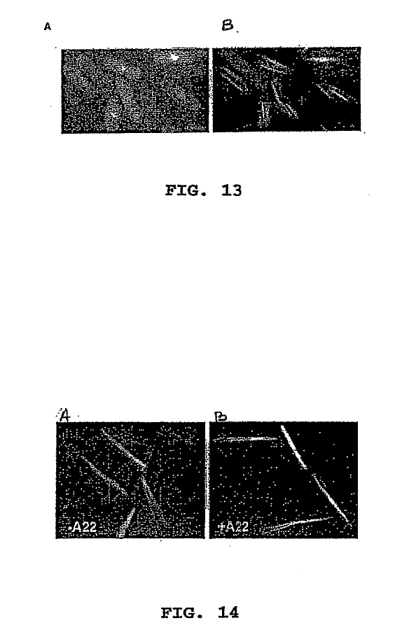

Next, whether MreB polymerization in fission yeast was mechanistically related to its polymerization in bacteria was assessed. Studies in bacteria have shown that the small molecule A22 affects the polymerization and/or maintenance of MreB polymers. To test whether A22 prevents polymerization of MreB in fission yeast, cells were treated with A22 prior to the induction of MreB expression. Cells expressing GFP-MreB were treated with the MreB-inhibitor A22 (0 minute time point) at a concentration of 50 μg/ml. The drug then was washed out, and cells were imaged by time-lapse microscopy. Cells treated with A22 did not exhibit MreB polymers. See FIG. 13A. The effect of A22 on MreB polymerization was reversible because removal of A22 resulted in rapid establishment of MreB filaments (FIG. 13B; 20 minutes). This established that A22 prevented MreB polymerization. Although A22 has been used in several bacteria, the precise mechanism of action of this compound (i.e., whether A22 prevents MreB polymerization or promotes disassembly of MreB polymers or both) is not known.

Having established that A22 prevented polymerization of MreB in fission yeast, whether A22 promoted severing and/or disassembly of preformed MreB bundles we studied. To this end, cells containing preformed MreB bundles were treated with A22. Cells displaying prominent GFP-MreB bundles (see FIG. 14 (−A22)) were treated with A22. Despite culturing of the cells for 1 hour with A22, pre-existing bundles were unaffected. See FIG. 14. The pre-existing MreB bundles were neither severed nor disassembled upon exposure to A22 suggesting that A22 affects MreB function by preventing its assembly.

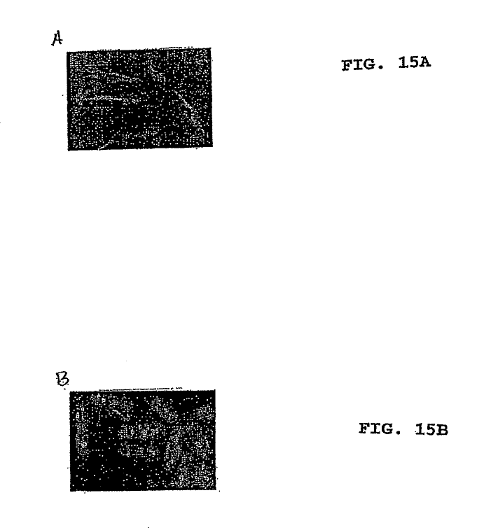

In bacteria, mutations in the vicinity of the ATP binding pocket of MreB confer A22 resistance. Two different mutations in the ATP binding region resulted in insensitivity to A22. Plasmids expressing GFP-MreB E143A (see Garner et al., Science 306:1021-1025, 2004) or D165E (see Kruse et al., EMBO J. 22:5283-5292, 2003) were introduced into wild type fission yeast cells. Cultures were grown in the absence of thiamine to induce the expression of GFP fusion proteins. The mutants assembled similar structures both in the presence and absence of A22. See FIGS. 15A (E143A) and 15B (D165E). These ATPase mutants assembled scrambled and qualitatively thinner polymers and/or large aggregates, suggesting that the ATPase activity of MreB is important for its normal organization in S. pombe. The ability of A22 to prevent assembly of MreB in fission yeast and the resistance of ATPase mutants to A22 treatment also suggests that MreB polymerization in fission yeast occurs by mechanisms similar to those in bacterial cells.

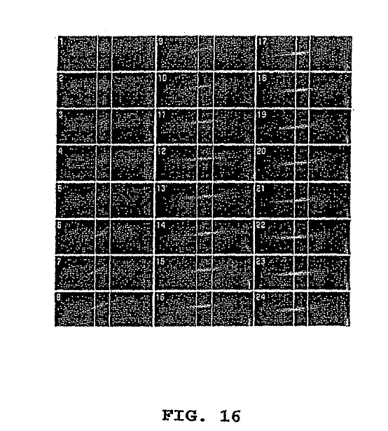

The effect of A22 treatment was reversible (see FIG. 13). Therefore A22 treatment and washout was used to study-the mechanisms of MreB polymerization. Cells expressing GFP-MreB were treated with A22. Subsequently, A22 was removed and cells were imaged by time-lapse microscopy, with images taken at 2-minute intervals. See FIG. 16. The vertical lines in each panel identify the position of the nucleus. The data indicate a roughly symmetrically bidirectional growth. Upon removal of A22, over 80% of the MreB nucleation events (n>40) occurred in the vicinity of the nuclear envelope and rapidly organized into a linear array. It is possible that the perceptibly higher concentration of GFP-MreB in the vicinity of the nucleus might be responsible for this effect. Growth of MreB bundles occurred in a bidirectional manner, with an average growth rate (n=44) of 0.46±0.03 μm/min, and did not vary significantly in the simultaneous presence of LatA and MBC.

### Example 6

### Fluorescence Recovery After Photobleaching

In order to further assess the properties of the polymers, fluorescence recovery after photo-bleaching (FRAP) analysis of the growing MreB bundles was carried out. A Zeiss Meta 510™ confocal microscope, with an apochromat 100×/1.25 NA oil-immersion objective, was used for confocal microscopy and fluorescence recovery after photobleaching (FRAP) experiments. Bleaching of the marked region was carried out with 100% laser power with 200 iterations. Z stack images were acquired at 55-second intervals. Fluorescence recovery was analyzed with Zeiss 3D for LSM™ software. Unbleached regions in the same filament were used to calculate the intensities of unbleached filament. The time taken from the point of nucleation of the filament to growth up to 3 μm was measured for calculating the filament growth rates.

Creating bleach marks on the MreB bundles using a laser beam (FIG. 17), followed by analysis of the growth of the polymers on either side of the bleached region, showed that the MreB bundles grew symmetrically in both directions. The bleached region of the bundle is shown with a white box, and the unbleached area is shown with a darker box in FIG. 17. Time-lapse images after photobleaching were acquired at 55-second intervals. Percentage of growth of GFP-MreB bundles on either side (arbitrarily referred to as left- or right-hand side of the bleach mark) of the bleached region is shown in FIG. 18 (n=18). Measurements were made on 18 such bundles. This suggests that MreB bundles grow in a symmetrically bidirectional manner with no obvious polarity in growth.

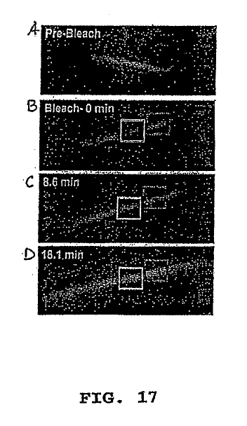

When bleach marks were generated on MreB bundles using a laser beam and the fluorescence intensities and the unbleached regions were followed over time by time-lapse microscopy, MreB monomers and/or protofilaments were added along the entire length of the growing bundles. Recovery of fluorescence in arbitrary units in the unbleached and-bleached regions, measured from 15 independent filaments, is shown in FIG. 19. The error bars represent the standard error.

Fluorescence-recovery profiles of the MreB bundles showed that the fluorescence intensities gradually increased all over the bundle length, with recovery occurring in both the bleached and the unbleached regions. See FIGS. 17, 19, and 20. This suggests that subunits were being added along the entire length of the polymer during the growth. However, it is currently unclear whether MreB monomers were added to the bundles or whether short protofilaments associate laterally with pre-existing filaments to form these stiff linear bundles.

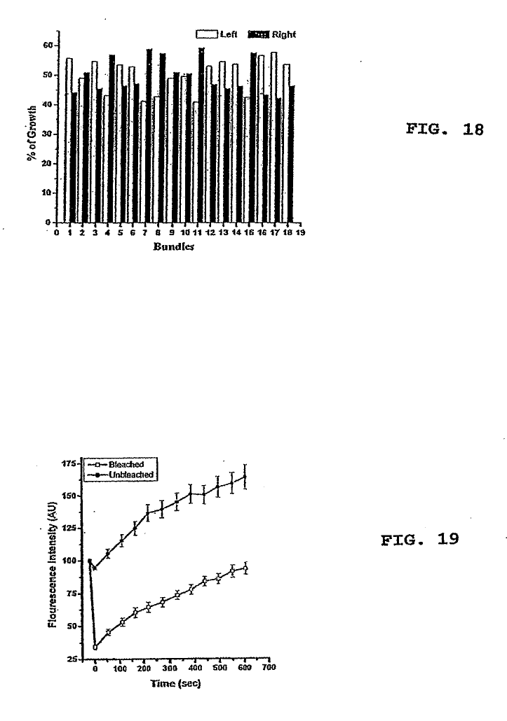

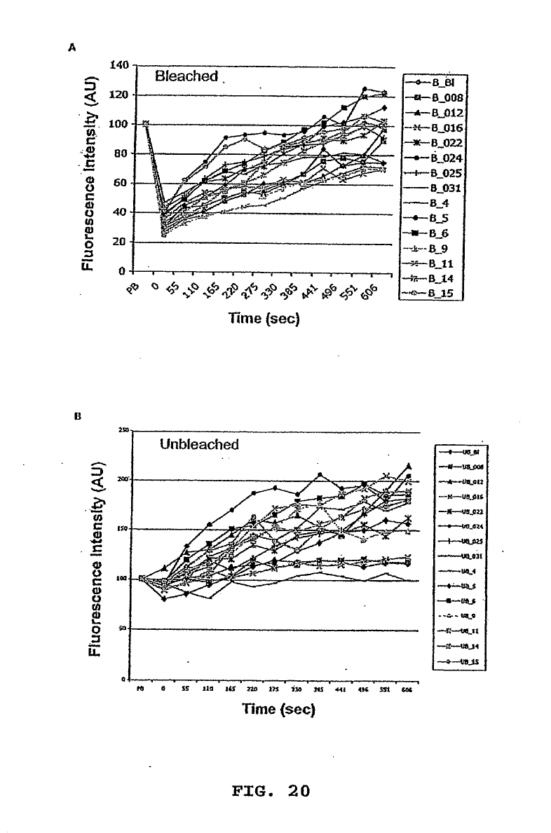

The fluorescence recovery of individual GFP-MreB bundles used in the FRAP experiments is quantitated in FIGS. 18 and 19. FIG. 20A shows data for a bleached region, and FIG. 20B shows data for an unbleached region. For these experiments, cells were grown in the presence of A22 and were mounted onto agarose slides after washing out A22. Cells with short MreB bundles were bleach-marked with a laser. Their growth and fluorescence recoveries were monitored immediately thereafter by acquiring Z stack images at a regular interval of 55 seconds. Fluorescence recovery was analyzed with Zeiss™ 3D for LSM software. Fluorescence intensities are plotted in FIG. 20 in arbitrary units measured in 15 independent filaments.

### Example 7

### Growth Dynamics for MreB Bundles in Fission Yeast

MreB organizes into spiral structures in E. coli cells. ParM and certain versions of SopA organize into linear structures, like MreB expressed in fission yeast. However, the mechanism of alignment of these polymers along the cellular long axis has not been investigated. To further understand the mechanism by which MreB aligns with the cellular long axis, time-lapse microscopy was used to image cells expressing GFP-MreB. Cultures carrying the GFP-MreB plasmid were grown in the absence of thiamine. A22 was added to the cultures at a final concentration of 50 μg/ml after 16 hours of growth. Cultures were further grown for a period of 8 hours after the addition of A22. A22 was subsequently washed out, and the cells were mounted onto agarose slides and immediately imaged by time-lapse microscopy. A time-lapse series shows the formation of MreB arrays after release from A22. treatment. See FIG. 21.

The MreB polymers, at the time of inception, had no specific orientation and were seen to be even perpendicular to the long axis of the cell (FIG. 21A, 10.5 minutes). As polymers grow, alignment along the long axis is established by sliding of bundles along the sides of the cell. Therefore, the longitudinal orientation of the MreB bundles appeared to arise by sliding of these bundles along the cortex of the cylindrical part of the cell during the growth of the bundle,. See FIG. 21A. Arrowheads point to the contact of bundles with the cell cortex. In the course of these time-lapse studies, two or more linear polymers of MreB often bundled together and continued to elongate. In most cases, bundling was initiated after contact of the end of one polymer with the end of another polymer.

FIG. 21B shows that MreB polymers bundle to form rigid polymers that then grow as a single entity. Arrowheads point to MreB's linear polymers that are seen to undergo lateral bundling. These studies revealed the ability of MreB to form lateral bundles and established that sliding along the cortex, as has been noticed for fission-yeast microtubules, contributes to longitudinal alignment of these MreB bundles.

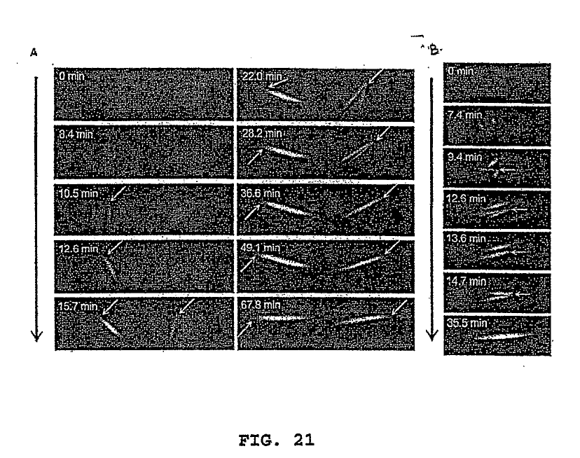

T shaped cells (tea1Δ) carrying pREP42-gfp-mreB plasmid were grown at 24° C. for 24-30 hours to allow expression of GFP-MreB and then shifted to 36° C. for 4 hours. Cultures were methanol-fixed and stained with antibodies against tubulin. The organization of multiple linear arrays of microtubules and GFP-MreB running across cell ends is shown in FIG. 22. In all cases studied, MreB bundles ceased to grow upon contact with the cortex around the hemispherical ends of the cell. See FIGS. 4, 7B and 22. Cessation of growth was particularly clear in teal mutants, wherein the bundles were found to run between each pair of cell ends, but were never found to curve at the hemispherical ends (FIG. 22). Failure of MreB bundles to curve at the cell ends was observed in wild-type (FIG. 4) and teal cells (FIG. 22), as well as in wee1-50 cells, which divide at a reduced size (FIG. 23).

### Example 8

### Assembly of MreB into Linear Polymers in Cells Lacking Microtubule-Organizing Proteins

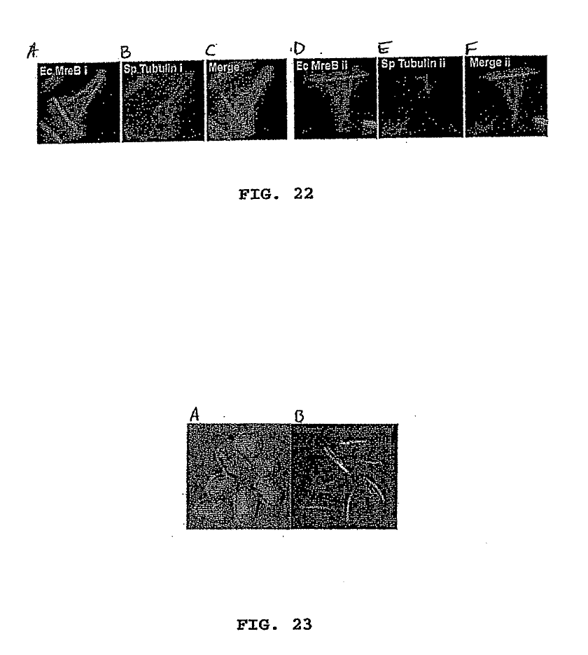

Whether F-actin or microtubule-organizing proteins were involved in the assembly of MreB into linear polymers was tested. To this end, GFP-MreB was expressed in mutants defective in the arp2/3 complex (see McCollum et al., EMBO J. 15:6438-6446, 1996 and Morrell et al., Mol. Biol. Cell 10:4201-4215, 1999), formins cdc12 and for3 (see Nurse and Thuriaux, Genetics 96:627-637, 1980 and Felerbach and Chang, Curr. Biol. 11:1656-1665, 2001), and the γ-tubulin complex protein alp4 (see Vardy and Toda, EMBO J. 19:6098-6111, 2000) and visualized MreB bundles at restrictive temperatures. All mutant strains carrying the GFP-MreB plasmid were grown at 24° C. for 24-30 hours to allow sufficient expression of MreB. However, at this stage, MreB polymers were still not assembled. Subsequently, cultures were shifted to 36° C. and further grown for a period of 4 hours. Cultures were fixed with 3.7% formaldehyde and stained either for actin (Rhodamine-phalloidin) or for tubulin (with TAT1 antibodies). GFP-MreB bundles (green) assembled in cells defective in the actin-nucleating factors Arp2/3p, Cdc12p (formin) or For3p (formin) or the γ-tubulin complex protein Alp4p are shown in FIG. 24. Actin or microtubules are shown in red. The bar represents 3 μm. MreB-assembly was largely unaffected in these mutants (FIG. 24). These experiments established that MreB assembly was independent of the tested yeast cytoskeleton-organizing proteins.

To test whether MreB bundles stop growth upon reaching the hemispherical region of the cell, wee1-50 cells expressing GFP-MreB were grown in the absence of thiamine at 24° C., shifted to 38° C. for 4 hours and images acquired. GFP-MreB bundles were found to run from one cell end to the other, but were not found to curve at the ends of the cell. See FIG. 23.

In fission yeast, the dynamic instability of microtubules ensures catastrophe upon contact of the growing microtubule with the cell ends, thereby preventing curving of microtubules at the cell ends. Given that E. coli ParM exhibits dynamic instability, it seemed likely that cessation of growth of MreB at the cell ends was mediated by potential dynamic instability of MreB bundles. To test whether MreB bundles underwent dynamic instability, wee1-50 cells expressing GFP-MreB were imaged. The wee1-50 cells expressing GFP-MreB were cultured in the presence of A22 (50 μg/ml) at 24° C. and then shifted to 38° C. for 4 hours. A22 was then washed off and mounted onto agarose slides and images were acquired.

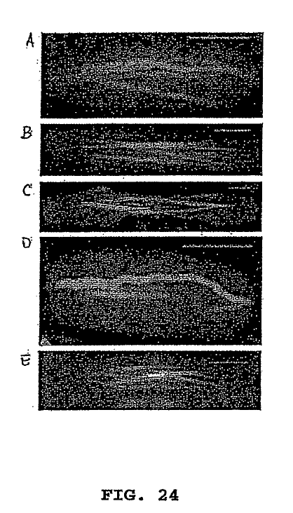

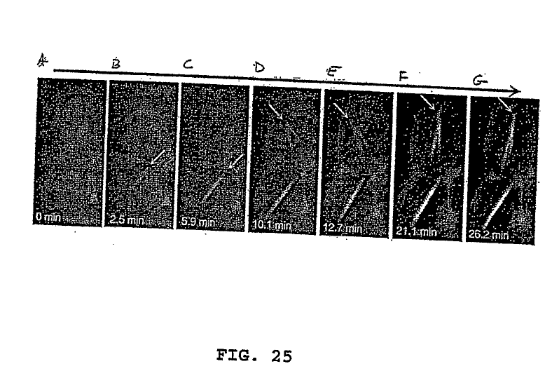

MreB polymerization stopped upon reaching the hemispherical ends of the cell. MreB bundles grew to the cell ends and ceased to elongate further, but did not display shrinkage upon reaching the hemispherical part of the cell (FIG. 25). The intensity of GFP-MreB along the sides continued to increase (by 40% form 10 minutes to 26 minutes) even after the ends of the bundles ceased growth, ruling out the possibility that unavailability of GFP-MreB contributed toward the instability fo MreB bundles to grow after reaching the cell ends. Thus it is possible that the hemispherical ends of the cell might provide physical barriers for insertion of monomers and/or short protofilaments at the ends of the stiff MreB bundles.

### Example 9

### MreB Organization in the Spherical orb6-25 Mutant

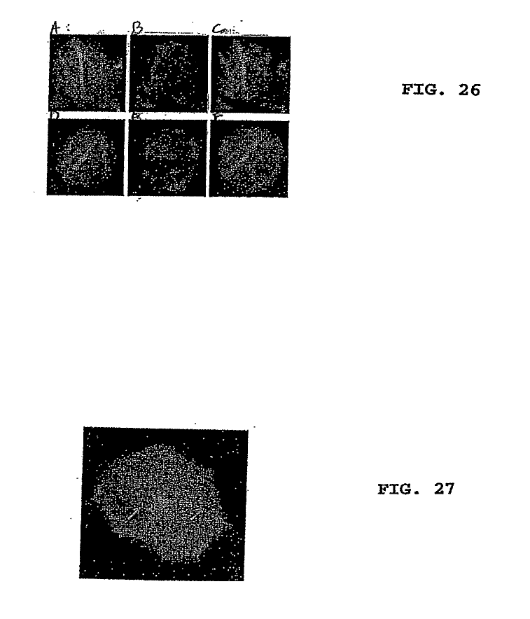

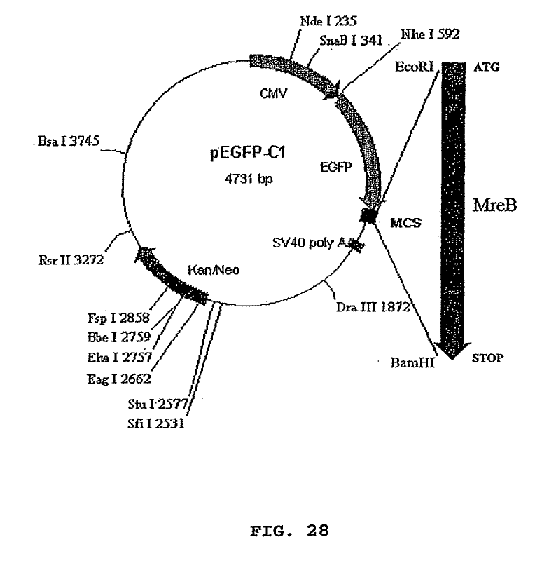

The organization of MreB polymers along the long axis of the fission-yeast cell suggested that the cylindrical cell morphology might contribute to such organization. To address this question, we tested the organization of MreB in the spherical orb6-25 mutant (see Verde et al., Proc. Natl. Acad. Sci. USA 95:7526-7531, 1998). Cultures of orb6-25 mutants carrying pREP41-gfp-mreB were initially grown at 24° C. for 24-30 hours and then shifted to 36° C. for 4 hours before fixation. The cells, expressing GFP-MreB, were fixed with methanol and stained with antibodies against tubulin. The organization of scrambled arrays of microtubules and GFP-MreB in orb6-25 is shown in FIG. 26. MreB polymers were found to be scrambled and were not organized in parallel arrays in these spherical cells. Collectively, these studies indicated that the assembly of MreB into a linear array in fission yeast requires a pre-existing cylindrical cell shape and is independent of F-actin and microtubules.

### Example 10

### Expression of GFP-MreB in Mammalian Cells

Having observed the organization of MreB into linear arrays in fission yeast, we tested whether MreB assembled into similar polymeric structures in mammalian cells. Transient expression of a GFP-MreB fusion from the CMV promoter in normal rat kidney (NRK) cells showed that MreB did assemble into filaments. See FIG. 27. Arrowheads indicate MreB filaments. However; the lower density of filaments might have been due to the lower-expression levels and concentration of MreB in mammalian cells as assessed by immunoblotting with GET antibodies.

The MreB gene was PCR-amplified from E. coli genomic DNA and digested:with the restriction enzymes EcoRI and BamHI. This PCR product was ligated to the pEGFP-Cl plasmid digested with the same enzymes to obtain the eGFP-MreB fusion construct for expression in mammalian cells. See FIG. 28.

### Example 11

### Screening Compound Libraries Using Identified Bacterial Protein Expressed in Yeast

A bacterial genomic DNA library is constructed in a yeast expression vector such as pREP42, tagged with GFP or another reporter. Yeast transformants expressing the bacterial genes as a GFP fusion are microscopically screened for clones that show any visible higher order assemblies or structures in the yeast cells. Proteins that exhibit any such assemblies or structures are assessed further for their function and whether they are essential in bacterial cells by expressing their gene as a GFP fusion in bacteria and then knocking out that gene function. If the gene is found to be essential, a chemical library of small molecules is screened for compounds that inhibit the activity of the protein. Inhibition of this activity is monitored using fluorescence microscopy and/or by monitoring assembly or lack of assembly in yeast. The compounds then are tested for toxicity on bacterial cultures. Compounds which are toxic to bacteria but not to eukaryotes would form a new class of antibacterial agents.

### Example 12

### Method for Identifying Antibacterial Target Proteins

A GFP fusion genomic library of a bacterium of interest is provided in a yeast expression vector and expressed in a convenient yeast cell. These yeast cells expressing the library are observed by fluorescence microscopy to identify GFP-labeled proteins in the library that form visible assemblies. These identified proteins then are assessed with respect to whether they are essential to the bacterial cell function. Those bacterial proteins which form visible assemblies and are essential to bacterial cell function are identified as antibacterial target proteins.

1. An in vitro method for identifying antibacterial compounds that have significantly greater toxicity to a specific bacterial protein than to eukaryotic cells, which comprises:
(a) providing a eukaryotic cell culture comprising eukaryotic cells;
(b) transfecting said cells with a gene encoding said bacterial protein;
(c) expressing said bacterial protein in said cells;
(d) screening said transfected eukaryotic cells with at least one candidate antibacterial compound for toxic effects against or inhibition of said bacterial protein and simultaneously assessing said transfected eukaryotic cells for indications of toxicity against eukaryotic cells; and
(e) identifying a compound that exhibits selective toxic effect against said specific bacterial protein.

2. The method of claim 1 wherein said eukaryotic cells are yeast cells.

3. The method of claim 2 wherein said yeast cells are Schizosaccharomyces pombe.

4. The method of claim 1 wherein said eukaryotic cells are mammalian cells.

5. The method of claim 4 wherein said mammalian cells are normal rat kidney cells.

6. The method of claim 1 wherein said bacterial protein is selected from the group consisting of FtsZ, MreB and both FtsZ and MreB.

7. The method of claim 1 wherein said bacterial protein is a fusion with a reporter protein.

8. The method of claim 7 wherein said reporter protein is Green Fluorescent Protein.

9. An antibiotic screening cell culture which comprises an in vitro culture of Schizosaccharomyces pombe that expresses a bacterial target protein selected from the group consisting of FtsZ, MreB, both FtsZ and MreB, and fusions thereof with a reporter protein.

10. The cell culture of claim 9 wherein said bacterial target protein is a fusion of FtsZ and Green Fluorescent Protein.

11. The cell culture of claim 9 wherein said bacterial target protein is a fusion of MreB and Green Fluorescent Protein.

12. A method of identifying antibacterial target proteins from bacteria, which comprises:
(a) providing d GFP fusion genomic library of a bacterium in a yeast expression vector;
(b) expressing said library in yeast cells;
(c) observing said yeast cells by fluorescence microscopy to identify GFP-labeled proteins of said expressed library that form visible assemblies;
(d) assessing said identified proteins with respect to whether said proteins are essential to cell function of said bacterium; and
(e) identifying proteins that form visible assemblies and are essential to cell function of said bacterium as antibacterial target proteins.

13. The method of claim 12 wherein said yeast cells are Schizosaccharomyces pombe.
